(19) United States
(12) Patent Application Publication
(10) Pub. No.: US 2021/0205382 A1
(43) Pub. Date: Jul. 8, 2021

(54) METHODS AND COMPOSITIONS FOR USING METAL ELEMENTS IN AAV GENE THERAPY

(71) Applicant: University of Florida Research Foundation, Incorporation, Gainesville, FL (US)

(72) Inventors: Arun Srivastava, Gainesville, FL (US); Himanshu K. Rambhai, Gainesville, FL (US)

(73) Assignee: University of Florida Research Foundation, Incorporation, Gainesville, FL (US)

(21) Appl. No.: 17/055,972

(22) PCT Filed: May 17, 2019
(86) PCT No.: PCT/US2019/032933

§ 371 Date: Nov. 16, 2020

Related U.S. Application Data
(60) Provisional application No. 62/839,395, filed on Apr. 26, 2019.
(60) Provisional application No. 62/833,610, filed on Apr. 12, 2019.
(60) Provisional application No. 62/673,085, filed on May 17, 2018.

(51) Int. Cl. A61K 35/761; C12N 15/86; C12N 7/00; A61K 33/30

(52) U.S. Cl. CPC A61K 35/761; A61K 33/30; C12N 7/00; C12N 15/86

(57) ABSTRACT

Disclosed herein are compositions and methods of improving transduction efficiency of rAAV administered to a subject using essential metal elements.

### RELATED APPLICATIONS

This application claims the benefit under 35 U.S.C. § 119(e) from U.S. provisional application No. 62/673,085, filed May 17, 2018, U.S. provisional application No. 62/833,610, filed Apr. 12, 2019, and U.S. provisional application No. 62/839,395, filed Apr. 26, 2019, the entire contents of each of which are incorporated by reference herein.

### GOVERNMENT SUPPORT

This invention was made with government support under HL-097088 awarded by the National Institutes of Health. The government has certain rights in the invention.

### BACKGROUND

Adeno-associated viruses (AAVs) are gaining ground as vectors for gene therapy applications, with numerous recombinant AAV (rAAV) being tested in clinical trials and many more in pre-clinical development. Compositions and methods to increase the efficiency of gene therapy involving rAAV would therefore be broadly useful.

### SUMMARY

AAVs are proving to be versatile gene delivery vectors for various diseases and conditions because not only do they allow infection of a broad variety of cells and are able to stably integrate into the host cell genome thus providing longevity of transgene expression, but they also present very low immunogenicity or other cytotoxic response. Improving the transduction efficiency of rAAV would be advantageous at least for the reason that a lower concentration of rAAV having a greater transduction efficiency would be required to be administered to a subject in need of gene therapy compared to rAAV with a lower transduction efficiency. The present disclosure is based, at least in part, on the surprising recognition that essential metal elements play a vital role in the transduction of transgenes in cells infected with rAAV comprising the transgenes, and that metal elements are able to improve the transduction efficiency of rAAV in cells infected with them. This disclosure is also based on the recognition that this effect of improved transduction efficiency of rAAV particles by metal elements may be more pronounced in cells having a reduced expression or quantity of functional metal ion transporters (e.g., ZIP14 for transporting Zn ions). Accordingly, provided herein are compositions and methods involving combining administration of rAAV with administration of essential metal elements to a subject such that administration of essential metal elements improve the transduction efficiency of administered rAAV.

In some aspects, provided herein is a method comprising administering to a subject a rAAV, and administering to the subject an essential metal element. In some embodiments, a subject is administered an amount of essential metal element in an amount that is at least equal to a target blood concentration multiplied by the total volume of the subject's blood. In some embodiments, a subject is administered an amount of essential metal element in an amount that results in increased AAV transduction efficiency.

In some embodiments, a subject is administered 2-5 essential metal elements.

In some embodiments, an essential metal element is selected from the group consisting of: calcium, magnesium, sodium, chromium, copper, cobalt, iron, manganese, molybdenum, zinc, and nickel. In some embodiments, an essential metal element is selected from the group consisting of: magnesium, zinc, cobalt, and nickel. In some embodiments, an essential metal element administered to the subject is zinc. In some embodiments, an essential metal element/s administered to the subject comprises zinc. In some embodiments, an essential metal element/s administered to the subject comprises zinc and cobalt. In some embodiments, an essential metal element/s administered to the subject comprises zinc and nickel. In some embodiments, an essential metal element/s administered to the subject comprises zinc and magnesium. In some embodiments, an essential metal element is provided in the form of a chloride salt.

In some embodiments, a subject is administered magnesium and the target blood concentration is approximately 26 μg/ml. In some embodiments, a subject is administered cobalt and the target blood concentration is approximately 12 μg/ml. In some embodiments, a subject is administered nickel and the target blood concentration is approximately 9 μg/ml. In some embodiments, a subject is administered zinc and the target blood concentration is approximately 14 μg/ml.

In some embodiments, a subject is administered an amount of essential metal element that is at least sufficient to achieve a target blood concentration of that element is the product of the target blood concentration, the total blood volume of the subject, and a factor that accounts for absorption, distribution, metabolism and elimination or excretion (ADME) of the essential metal element as provided. In some embodiments, a factor which accounts for ADME is 1-100.

In some embodiments, more than one essential metal elements (e.g., 2, 3, 4, 5, 6, 7, 8, 9, 10, or 11 or more) are administered simultaneously. In some embodiments, more than one essential metal elements (e.g., 2, 3, 4, 5, 6, 7, 8, 9, 10, or 11 or more) are comprised in a single composition.

In some embodiments, rAAV is administered simultaneously with the essential metal element/s. In some embodiments, rAAV and essential metal element/s are comprised in a single formulation. In some embodiments, the essential metal element/s is administered to the subject after administering the rAAV. In some embodiments, essential metal element/s is administered to the subject at least a second time after administering the rAAV. In some embodiments, an essential metal element/s is administered to the subject 2-5 times after administering the rAAV. In some embodiments, an essential metal element/s is administered to the subject at least one minute after administering the rAAV. In some embodiments, an essential metal element/s is administered to the subject at least one hour after administering the rAAV. In some embodiments, an essential metal element/s is administered to the subject at least one day after administering the rAAV. In some embodiments, an essential metal element/s is administered to the subject at least one week after administering the rAAV. In some embodiments, a subject is administered an essential metal element/s the second time at least a week after administering an essential metal element/s to the subject the first time.

In some embodiments, an essential metal element/s is administered to the subject enterally. In some embodiments, an enteral administration of the essential metal element/s is oral. In some embodiments, an essential metal element/s is administered to the subject parenterally. In some embodiments, an essential metal element is administered to the subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, an essential metal element/s is administered to the subject by injection into the hepatic artery or portal vein.

In some embodiments, rAAV is single stranded or self-complementary. In some embodiments, the serotype of the rAAV is 1, 2, 3, 4, 5, 6, 7, 8, 9, 10, 11, 12, or 13. In some embodiments, rAAV is a pseudotype. In some embodiments, rAAV comprises a chimeric rep gene, or chimeric capsid protein.

In some embodiments, rAAV comprises a therapeutic gene. In some embodiments, a therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, a Fc-fusion protein, an anticoagulant, a nuclease, guide RNA or other nucleic acid or protein for gene editing.

In some embodiments, a subject is a mammal. In some embodiments, a mammal is a human. In some embodiments, a subject is an adult human, and the total blood volume is approximately 5 liters. In some embodiments, a subject is an adult human male, and the total blood volume is determined using Nadner's equation as ((0.006012×Height³)/(14.6×Weight))+604, wherein the height is entered in inches and the weight is entered in pounds. In some embodiments, a subject is an adult human female, and the total blood volume is determined using Nadner's equation as ((0.005835×Height³)/(15×Weight))+183, wherein the height is entered in inches and the weight is entered in pounds. In some embodiments, a mammal is a mouse, and the total blood volume is approximately 2 ml.

In some embodiments of any one of the methods disclosed herein, administering an essential metal element/s results in a 1.5-20-fold increase in AAV transduction efficiency when compared to when AAV is administered alone. In some embodiments, a fold increase in AAV transduction efficiency is measured by infecting test cells in an in vitro culture with AAV with and without essential metal elements.

In some aspects, provided herein is a method comprising administering to a subject that has received a rAAV an essential metal element. In some embodiments, a subject is administered an amount of essential metal element in an amount that is at least equal to a target blood concentration multiplied by the total volume of the subject's blood. In some embodiments, a subject is administered an amount of essential metal element in an amount that results in increased AAV transduction efficiency.

In some aspects, provided herein is a method comprising administering to a subject who has received a rAAV and an essential metal element the essential metal element a second time. In some embodiments, a subject is administered an amount of essential metal element in an amount that is at least equal to a target blood concentration multiplied by the total volume of the subject's blood. In some embodiments, a subject is administered an amount of essential metal element in an amount that results in increased AAV transduction efficiency.

In some aspects, provided herein is a pharmaceutical composition comprising a rAAV, and one or more essential metal elements. In some embodiments, the ratio of rAAV and one or more essential metal elements in a pharmaceutical composition results in increased AAV transduction efficiency when administered to a subject, or in a target blood concentration/s of the one or more essential metal elements. In some embodiments, a pharmaceutical composition comprises a pharmaceutically acceptable carrier.

In some aspects, provided herein is a method comprising administering to a subject a recombinant adeno-associated virus (rAAV) particle, and administering to the target tissue or organ an essential metal element in an amount that is sufficient to result in a target extracellular concentration of 1-50 μg/ml of the essential metal element in the target tissue or organ. In some embodiments, rAAV particles are administered to the target tissue or organ. In some embodiments, a target extracellular concentration is around 10 μg/ml, 20 μg/ml, 30 μg/ml, or 40 μg/ml.

In some aspects, provided herein is a method comprising administering to a subject an essential metal element, wherein the essential metal element is administered in an amount that is sufficient to result in a target extracellular concentration of 1-50 μg/ml of the essential metal element in the target tissue or organ, or in an amount that is at least equal to a target blood concentration multiplied by the total volume of the subject's blood. In some embodiments, a subject has cancer. In some embodiments, a metal element is administered to a target tissue or organ. In some embodiments, a target tissue is cancer tissue. In some embodiments, a metal element is administered directly to cancer tissue in the subject.

In some embodiments, a method comprising administering to a subject an essential metal element further comprises administering to a subject a recombinant adeno-associated virus (rAAV) particle. In some embodiments, a rAAV particle comprises a nucleic acid encoding a therapeutic gene. In some embodiments, a therapeutic gene targets a cancer.

In some embodiments of a method comprising administering to a subject an essential metal element, a subject is administered 2-5 essential metal elements. In some embodiments, the essential metal element is selected from the group consisting of: calcium, magnesium, sodium, chromium, copper, cobalt, iron, manganese, molybdenum, zinc, and nickel. In some embodiments, the essential metal element is selected from the group consisting of: magnesium, zinc, cobalt, and nickel. In some embodiments, an essential metal element administered to the subject is zinc. In some embodiments, an essential metal element administered to the subject comprises zinc.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1K show the dose-dependency of enhanced green fluorescent protein (EGFP) reporter protein expression as a function of varying concentrations of essential metal elements in HeLa cells infected with self-complementary rAAV2 (scAAV2) comprising nucleic acid encoding EGFP (scAAV2-EGFP).

FIG. 1A shows EGFP expression as a function of concentration of copper chloride.

FIG. 1B shows EGFP expression as a function of concentration of manganese chloride.

FIG. 1C shows EGFP expression as a function of concentration of cobalt chloride.

FIG. 1D shows EGFP expression as a function of concentration of sodium chloride.

FIG. 1E shows EGFP expression as a function of concentration of calcium chloride.

FIG. 1F shows EGFP expression as a function of concentration of zinc chloride.

FIG. 1G shows EGFP expression as a function of concentration of iron oxide.

FIG. 1H shows EGFP expression as a function of concentration of magnesium chloride.

FIG. 1I shows EGFP expression as a function of concentration of nickel chloride.

FIG. 1J shows EGFP expression as a function of concentration of chromium oxide.

FIG. 1K shows EGFP expression as a function of concentration of molybdenum chloride.

FIGS. 2A-2B show increase in transduction efficiency of EGFP after treatment with one or more essential metal elements in HeLa cells infected with scAAV2-EGFP. Control cells were infected with scAAV2-EGFP but not treated with any essential metal elements. Mock cells were not infected with scAAV2-EGFP. Data in FIG. 2A and FIG. 2B are representative of two separate experiments.

FIG. 3 shows transduction efficiency of rAAV2-CMV-mCherry in Huh7 cells either overexpressing ZIP14 or mock-transfected for varying concentrations of zinc added to the culture media.

FIG. 4 shows percentage of cells transduced by scAAV3-EGFP in cells treated with varying concentrations of zinc chloride using flow cytometry.

FIG. 5 shows AAV transduction efficiency and cell viability data in CCK8 cells transduced with AAV2 comprising mCherry encoding nucleic acid and treated with varying concentrations of zinc.

FIG. 6 shows AAV transduction efficiency and cell viability data in HEK293 cells transduced with AAV2 comprising mCherry encoding nucleic acid and treated with varying concentrations of zinc.

FIG. 7 shows AAV transduction efficiency and cell viability data in HeLa cells transduced with AAV2 comprising mCherry encoding nucleic acid and treated with varying concentrations of zinc.

FIG. 8 shows AAV transduction efficiency and cell viability data in HepG2 cells transduced with AAV2 comprising mCherry encoding nucleic acid and treated with varying concentrations of zinc.

FIG. 9 shows Western blot analysis of Factor IX expression in HepG2 cells infected with AAV3 particles comprising nucleic acid encoding Factor IX gene, either with or without treatment with 20 μg/ml ZnCl₂.

### DETAILED DESCRIPTION

Metal elements are essential components of approximately half of all cellular proteins, and approximately one-third of all known enzymes are metallo-enzymes. Moreover, various diseases and conditions are known to involve faulty metal ion transport between cells and intercellular space. For example, numerous population studies have reported a significant decrease (55-75%) in Zn2+ levels in HCC tissues, compared with healthy liver tissues (Cancer Bio. & Ther., 15: 353-360, 2014, which is incorporated herein by reference in its entirety). Dysfunctional zinc signaling is associated with a number of chronic disease states as well including cancer, cardiovascular disease, Alzheimer's disease, and diabetes (see e.g., Myers et al., Zinc Transporters, Mechanisms of Action and Therapeutic Utility: Implications for Type 2 Diabetes Mellitus; Journal of Nutrition and Metabolism, Volume 2012, Article ID 173712, which is incorporated herein by reference in its entirety).

Since numerous cellular proteins impact infection by and replication of rAAV, and expression of transgenes delivered by rAAV (e.g., those used for gene therapy), the inventors of the present disclosure sought to evaluate the effects of essential metal elements on rAAV transduction efficiency. It was found unexpectedly found that the transfection efficiency of rAAV was significantly improved when essential metal elements were added to cell cultures. It was further found that treatment of cells with a combination of certain essential metal elements provides an unexpectedly high increase in transduction efficiency. In some embodiments, AAV transduction efficiency is the rate of expression of a transgene in or by a cell that is infected with rAAV comprising the transgene per the number of rAAV particles that have been introduced to a cell (e.g., a particular multiplicity of infection (MOI)). In some embodiments, AAV transduction efficiency is measured as the number of cells in a culture that expresses a certain level (e.g., visible under microscopy) of transgene introduced by infecting the cell with rAAV. Improvement in AAV transduction efficiency is a measure of AAV transduction relative to the condition wherein rAAV is administered without additional essential metal elements (e.g., additional to the base levels existing in cell culture media, or buffers use to prepare a pharmaceutical composition).

Accordingly, provided herein are compositions and methods useful for increasing the AAV transduction efficiency of rAAV administered to a cell, organoid, organ, tissue, or subject (e.g., for the purpose of delivering a transgene as a therapy, or as an imaging agent). Accordingly, provided herein is a method comprising administering to a subject a rAAV, and administering to the subject an essential metal element. In some embodiments, an essential metal element is administered in an amount that is at least sufficient to achieve a target blood concentration of the element in the subject. In some embodiments, an essential metal element is administered in an amount that is at least sufficient to achieve a target extracellular concentration of the element in the subject in a particular tissue (e.g., disease tissue, for example cancerous tissue, or other target tissue) or organ (e.g., liver or brain). In some embodiments, an essential metal element is administered in an amount that is at least sufficient to achieve a target tissue concentration of the element in the subject in a particular tissue (e.g., cancerous tissue) or organ (e.g., liver or brain). In some embodiments, an essential metal element is administered in an amount that is at least sufficient to result in increased AAV transduction efficiency (e.g., in the target tissue).

In some embodiments, provided herein is a method comprising administering to a subject who has received a rAAV an essential metal element. In some embodiments, provided herein is a method comprising administering to a subject who is being administered a rAAV an essential metal element. In some embodiments, an essential metal element can be administered to a subject prior to rAAV administration. Also provided herein is a method comprising administering to a subject who has received a rAAV and at least one administration of an essential metal element, a subsequent (e.g., second, third, fourth, second and third, or second, third, and fourth) essential metal element.

### Essential Metal Elements

In some embodiments, essential metal elements are those metal elements that are necessary for the maintenance of life. In some embodiments, essential metal elements are those metal elements the absence of which causes death or severe malfunction of bodily mechanisms. Maret provides a summary of essential metal elements in Int J Mol Sci. 2016 January; 17(1): 66, which is incorporated herein by reference in its entirety. Interrelations between essential metal elements and human diseases are discussed in “Interrelations between Essential Metal Ions and Human Diseases” edited by Sigel, Astrid, Sigel, Helmut, Sigel, and Roland (ISBN 978-94-007-7500-8), and Myers et al., Zinc Transporters, Mechanisms of Action and Therapeutic Utility: Implications for Type 2 Diabetes Mellitus; Journal of Nutrition and Metabolism, Volume 2012, Article ID 173712, each of which is also incorporated herein by reference in its entirety. In some embodiments, essential metal elements are bulk elements, which are those elements which are needed in higher concentrations in the body (e.g., sodium, potassium, magnesium, or calcium). In some embodiments, essential metal elements are trace elements, which are those elements which are needed in very low concentrations for bodily functions (e.g., manganese, iron, cobalt, copper, zinc, molybdenum, or selenium). In some embodiments, an essential metal elements is an ultra-trace element, which are proposed to be essential and required in very low amounts (e.g., chromium, vanadium, nickel, silicon, or arsenic). In some embodiments, an essential metal element is selected from the group consisting of: barium, calcium, magnesium, potassium, sodium, chromium, copper, cobalt, iron, manganese, molybdenum, selenium, vanadium, tungsten, silicon, tin, zinc, nickel, and arsenic. In some embodiments, an essential metal element administered to a subject is selected from the group consisting of: calcium, magnesium, sodium, chromium, copper, cobalt, iron, manganese, molybdenum, zinc, and nickel. In some embodiments, an essential metal element administered to a subject is selected from the group consisting of copper, manganese, magnesium, nickel, cobalt, and zinc. In some embodiments, an essential metal element administered to a subject is selected from the group consisting of magnesium, nickel, cobalt, and zinc. In some embodiments of any one of the methods disclosed herein, a subject is administered zinc. In some embodiments of any one of the methods disclosed herein, a subject is administered magnesium. In some embodiments of any one of the methods disclosed herein, a subject is administered nickel. In some embodiments of any one of the methods disclosed herein, a subject is administered cobalt.

In some embodiments of any one of the methods disclosed herein, a subject is administered more than one (e.g., two, three, four, five, six, seven, more than one, more than two, more than three, more than 4, or more than five) essential metal elements. For example, in some embodiments, a subject is administered magnesium and nickel. In some embodiments, a subject is administered magnesium and cobalt. In some embodiments, a subject is administered magnesium and zinc. In some embodiments, a subject is administered cobalt and nickel. In some embodiments, a subject is administered copper and magnesium. In some embodiments, a subject is administered copper and nickel. In some embodiments, a subject is administered copper and cobalt. In some embodiments, a subject is administered any one or more of any of the metal elements described herein but not one or more of the metal elements described herein. In some embodiments, any one or more of the essential metal elements disclosed herein is excluded from being administered to a subject. For example, a subject is administered zinc but not cobalt, or a subject is administered zinc and cobalt but not magnesium. In some embodiments, a method as disclosed herein consists of administering to a subject a recombinant adeno-associated virus (rAAV), and/or administering to the subject an essential metal element. In some embodiments, a method as disclosed herein consists essentially of administering to a subject a recombinant adeno-associated virus (rAAV), and/or administering to the subject an essential metal element.

In some embodiments of any one of the methods disclosed herein, a subject is administered zinc and at least another essential metal element (e.g., zinc and cobalt, zinc and magnesium, zinc and nickel, or zinc and copper). In some embodiments, a subject is administered zinc and cobalt. In some embodiments, a subject is administered zinc and nickel. In some embodiments, a subject is administered more than two essential metal elements (e.g., zinc, cobalt, and nickel; or zinc, magnesium, and nickel; or zinc, cobalt, and nickel; or magnesium, nickel, and cobalt; or zinc, magnesium, and copper).

In some embodiments of any one of the methods disclosed herein, a subject is administered nickel and at least another essential metal element (e.g., zinc, magnesium, cobalt, zinc and magnesium, or zinc and cobalt).

In some embodiments of any one of the methods disclosed herein, a subject is administered cobalt and at least another essential metal element (e.g., zinc, nickel, magnesium, nickel and zinc, or zinc and magnesium).

In some embodiments of any one of the methods disclosed herein, a subject is administered magnesium and at least another essential metal element (e.g., zinc, nickel, copper, nickel and zinc, or cobalt and zinc).

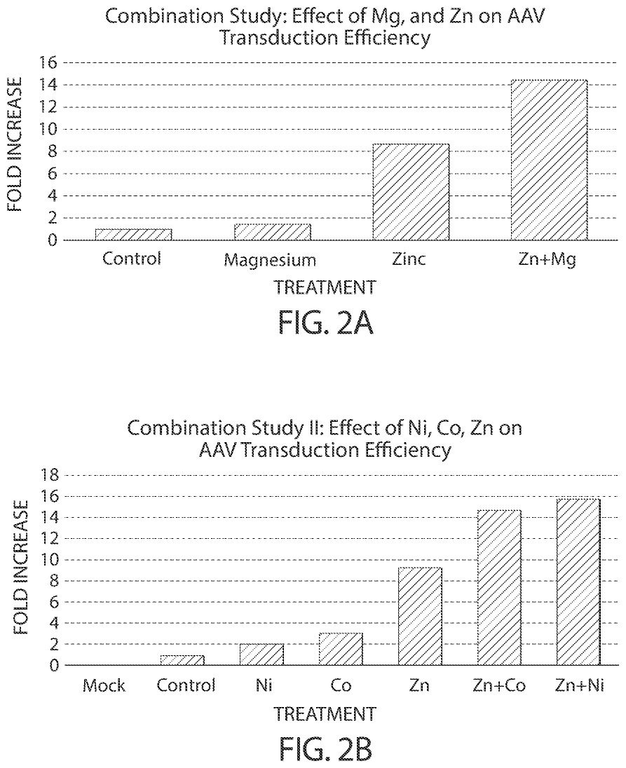

In some embodiments, administration of a combination of essential metal elements results in a synergistic effect that is more than additive of when a subject in administered each essential metal element alone. For example, while treatment of cells infected with rAAV with zinc alone improves AAV transduction efficiency by ˜8-fold and treatment of cells infected with rAAV with cobalt alone improves AAV transduction efficiency by ˜2-fold, treatment of cells with zinc and cobalt results in an AAV transduction efficiency of ˜15-fold. Another non-limiting example of a combination of essential metal elements resulting in an effect on AAV transduction efficiency that is more than additive is the combination of zinc and nickel, which results in almost ˜16-fold increase in AAV transduction efficiency, compared to only a ˜2-fold and ˜8-fold increase when cells are treated with nickel alone, and zinc alone, respectively. See for example, FIG. 2B.

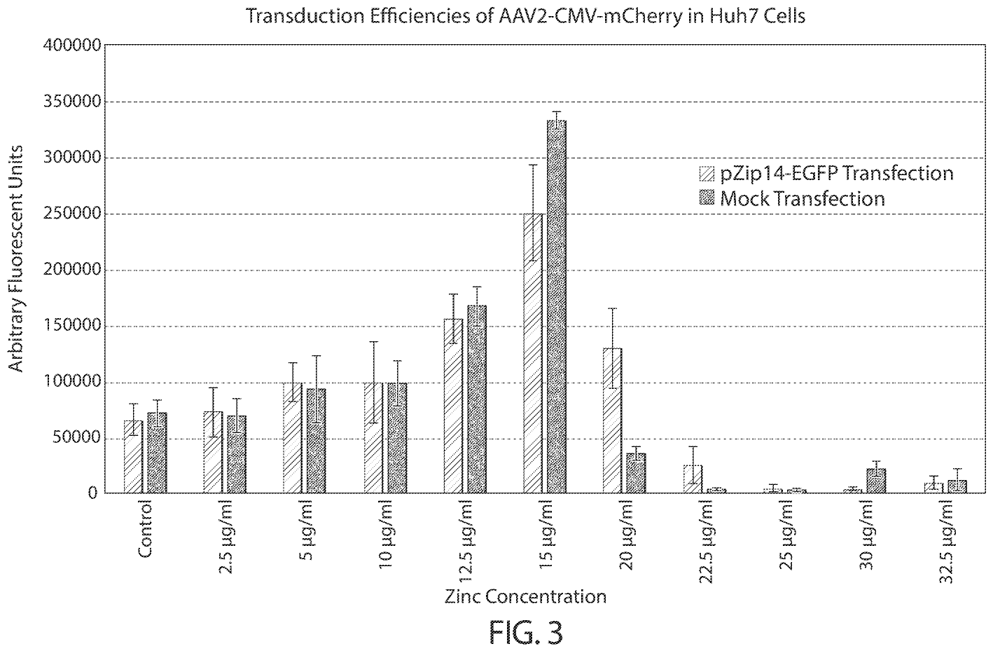

In some embodiments of any one of the methods disclosed herein, a second administration of essential metal element comprises a different essential metal element compared to the first administration of essential metal element to the subject. Such an approach may be adapted for numerous reasons, e.g., to keep levels of each essential metal element below toxic levels, while still achieving an improvement in AAV transduction. For example, a subject may be administered zinc in the first administration and cobalt in the second administration, or zinc and cobalt in the first administration, and zinc and nickel in the second administration. Other non-limiting approaches to keep levels of essential metal elements in the blood of subject below sub-toxic levels, and which are discussed below, are varying the quantities of essential metal elements administered, varying the timing between subsequent administrations of one or more essential metal elements, and varying route of delivery (e.g., delivering to a particular organ or tissue directly (e.g., by injection) rather than delivering systemically). Another non-limiting approach of limiting the toxicity of essential metal elements is to target tissues that have a greater tolerance for a particular metal element (e.g., because they have higher expression of one or more transporters that transport extracellular metal element into the cells). In some embodiments, metal element-induced toxicity may be beneficial. For example, cells involved in human hepatocellular carcinoma (HCC) are known to have a lower expression of zinc ion transporter (e.g., ZIP14), which may result in a lower tolerance for zinc (see e.g., FIG. 3), but allows zinc treatment-induced improvement of rAAV transduction efficiency. Toxicity in such cancer cells may not be undesired.

It is to be understood that an essential metal element can be provided in the form of any compound, e.g., in the form of a salt, for example such as a chloride, acetate, sulphate, nitrate, phosphate, molybdate, oxide, or chromate. Table 1 provides examples of different forms of various essential metal elements that may be administered to a subject to improve AAV transduction efficiency. Choice of the particular form of an essential metal element may depend on a number of factors, e.g., solubility of salt in a buffer suitable for administration of the element, route of administration, toxicity of particular form of metallic compound, target intracellular or extracellular metal element concentration, and/or tissue to be targeted. For example, if a tissue that is targeted is susceptible to toxicity of a particular element, administering the particular element may allow dosing with lower amounts to achieve the same level of improvement of rAAV transduction efficiency. For example, it has been found that HCC cells have a lower expression of ZIP14 zinc transporter due to which they manifest toxicity at a lower zinc concentration than when such cells are overexpressed with ZIP14 (see e.g., FIG. 3), and rAAV transduction efficiency for HCC cells is increased at lower zinc levels than for other cells that have higher levels of zinc transporter. In some embodiments, a bolus of essential metal element is provided by prescribing consumption of a food that is rich is that particular essential metal element.

Similarly, choice of particular essential metal element to be administered to a subject is made depending on its ability to improve AAV transduction efficiency, toxicity, tolerability for a short-term (non-chronic) dosing, pharmacokinetics and pharmacodynamics (e.g., including retention in liver), adsorption, distribution, metabolism, and excretion (ADME) properties, and/or ability to reach targeted tissue.

TABLE 1
Examples of essential metal elements and administration concentrations

| | | | Target Blood | | *Quantity |
|---|---|---|---|---|---|
| | | | Concentration | *Quantity | of Example |
| Essential | Target Blood | Example | of Example | of Essential | Compound to |
| Metal | Concentration | Compound to | Compound | Metal Element | Administer |
| Element | (μg/ml) | Administer | (μg/ml) | (mg) | (mg) |
| Zn | 14 | ZnCl₂ | 30 | 70 | 150 |
| | 14 | ZnSO4 | 35 | 71 | 175 |
| | 14 | C₁₂H₂₂O₁₄Zn | 100 | 72 | 500 |
| | | (Zinc | | | |
| | | Gluconate) | | | |
| Cu | 14 | CuCl₂ | 30 | 71 | 150 |
| Co | 12 | CoCl₂ | 25 | 58 | 125 |
| Mg | 26 | MgCl₂ | 100 | 128 | 500 |
| | 26 | MgO | 45 | 136 | 225 |
| Ni | 9 | NiCl₂ | 20 | 45 | 100 |

*For an adult human being having 5 L of blood; Not accounting for losses (i.e., without a factor accounting for ADME)

### Subjects

Aspects of the disclosure relate to methods for use with a subject. In some embodiments, a subject is a mammal. In some embodiments, a mammalian subject is a human, a non-human primate, a dog, a cat, a hamster, a mouse, a rat, a pig, a horse, a cow, a donkey or a rabbit. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is a laboratory animal, e.g., a mouse. In some embodiments, the subject is a human subject. In some embodiments, a subject is an adult (e.g., an adult human, or adult mouse). In some embodiment, a subject is juvenile (e.g., an infant, or a teenager).

In some embodiments, one or more essential metal elements are administered to a subject in need thereof. A subject in need of essential metal elements may be a subject who is in the need for gene therapy (e.g., a subject suffering from a disease of condition that is caused, or affected by, expression of one or more RNAs or proteins), and wherein the gene therapy is administered using rAAV.

In some embodiments, a subject that is administered one or more essential metal elements and has been or is being administered rAAV particles, has a disease or condition known to involve faulty or dysfunctional metal ion transport between cells and intercellular space (e.g., cancer, cardiovascular disease, Alzheimer's disease, and diabetes). In some embodiments, a subject that is administered one or more essential metal elements and has been or is being administered rAAV particles, has a disease or condition that has a compartmentalized tissue to be treated (e.g., a solid tumor).

### Administration and Dosing of Essential Metal Elements

In some embodiments, a subject is administered an essential metal element in an amount that is at least sufficient to achieve a target blood concentration of that element. In some embodiments, an amount that is at least sufficient to achieve a target blood concentration of that element is the product of the target blood concentration and the total blood volume of the subject. For example, to achieve a target blood concentration of 14 μg/ml of zinc in a subject having 5 liters of total blood volume, a total of (14 μg/ml)×(5 liters)=70 mg of zinc has to be administrated.

In some embodiments, an amount that is at least sufficient to achieve a target blood concentration of an essential element is the product of total blood volume and the difference between the target blood concentration and the existing blood concentration in the subject. For example, to achieve a target blood concentration of 14 μg/ml of zinc in a subject having 5 liters of total blood volume with an existing zinc concentration of 5 μg/ml, a total of (14-5 μg/ml)×(5 liters)=45 mg of zinc has to be administrated. Existing blood concentrations of blood can be estimated based on known average values for a subject of a particular species, sex, and age. In some embodiments, the existing blood concentrations of an essential metal element is determined by sampling and assaying the blood of a subject using methods known in the art.

In some embodiments, an amount that is at least sufficient to achieve a target blood concentration of that element is the product of the target blood concentration, the total blood volume of the subject, and a factor that accounts for absorption, distribution, metabolism and elimination or excretion (ADME) of the essential metal element as provided. For example, to achieve a total blood concentration of 14 μg/ml of zinc over 6 h after administration, (14 μg/ml)×(5 liters)×(1.2)=84 mg of zinc may have to be administrated. Pharmacokinetic studies of essential metal elements have been previously performed and methods of performing them are known also (see e.g., Eur J Drug Metab Pharmacokinet. 1991 October-December; 16(4):315-23; Am J Clin Nutr. 2017 December; 106(Suppl 6):1559S-1566S; Chemosphere. 2017 July; 178:513-533; Nutrients. 2016 Jul. 22; 8(7). pii: E444. doi: 10.3390/nu8070444; J Pediatr (Rio J). 2017 November-December; 93 Suppl 1:19-25. doi: 10.1016/j.jped.2017; Res Vet Sci. 2016 February; 104:106-12. doi: 10.1016/j.rvsc.2015.12.007, each of which are incorporated herein by reference in their entirety). These studies can be used to estimate the factor accounting for ADME of the particular essential metal element to be administered. In some embodiments, the factor which accounts for ADME depends on the route of administration. For example, the factor which accounts for ADME will be lower for delivery to the eye by an intraocular injection, rather than an oral or otherwise systemic delivery to the eye.

In some embodiments, the factor which accounts for ADME is 1-100 (e.g., 1-100, 1-3, 1.1-99.9, 1-5, 1-10, 5-10, 10-20, 10-30, 10-50, 20-50, 20-80, 50-80, 50-100, or 80-100). In some embodiments, the factor which accounts for ADME is greater than 100 (e.g., greater than 100, greater than 110, greater than 150, greater than 200, greater than 400, greater than 500, or greater than 1000). In some embodiments, the factor which accounts for ADME is 1-100 (e.g., 1, 2, 3, 4, 5, 6, 7, 8, 9, 10, 15, 20, 30, 50, 80, 90, or 100).

In some embodiments, target blood concentrations of an essential metal element can be achieved by administering to the subject an amount of the essential element that is 10-1000% (e.g., 10-20, 10-40, 10-5-, 20-100, 20-20, 20-50, 20-60, 20-80, 40-60, 40-80, 50-80, 50-100, 60-100, 80-100, 90-100, 95-100, 100-200, 200-300, 100-500, 200-500, 500-800, or 500-1000%) higher than the amount in the blood of a subject. In some embodiments, target blood concentrations of an essential metal element can be achieved by administering to the subject an amount of the essential element that is 10-1000% (e.g., 10-20, 10-40, 10-5-, 20-100, 20-20, 20-50, 20-60, 20-80, 40-60, 40-80, 50-80, 50-100, 60-100, 80-100, 90-100, 95-100, 100-200, 200-300, 100-500, 200-500, 500-800, or 500-1000%) higher than the target blood concentrations as determined by conducting in vitro experiments.

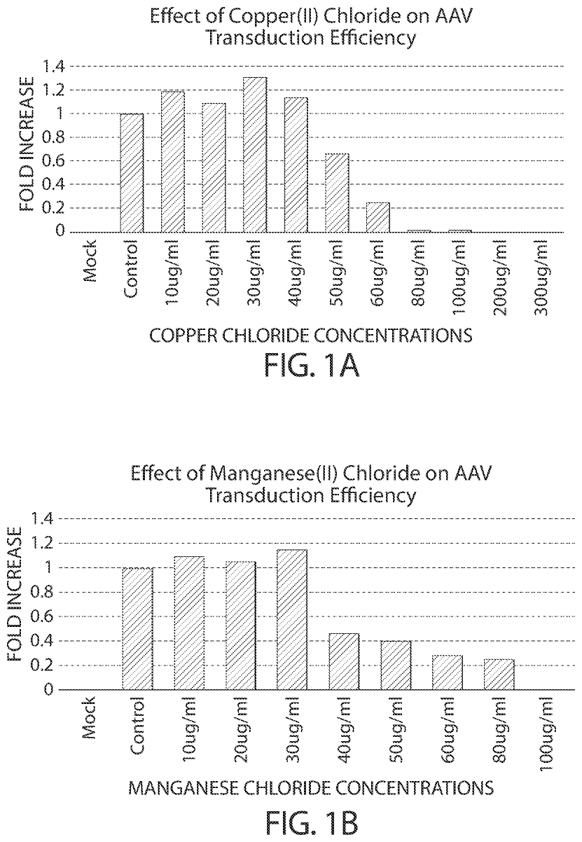

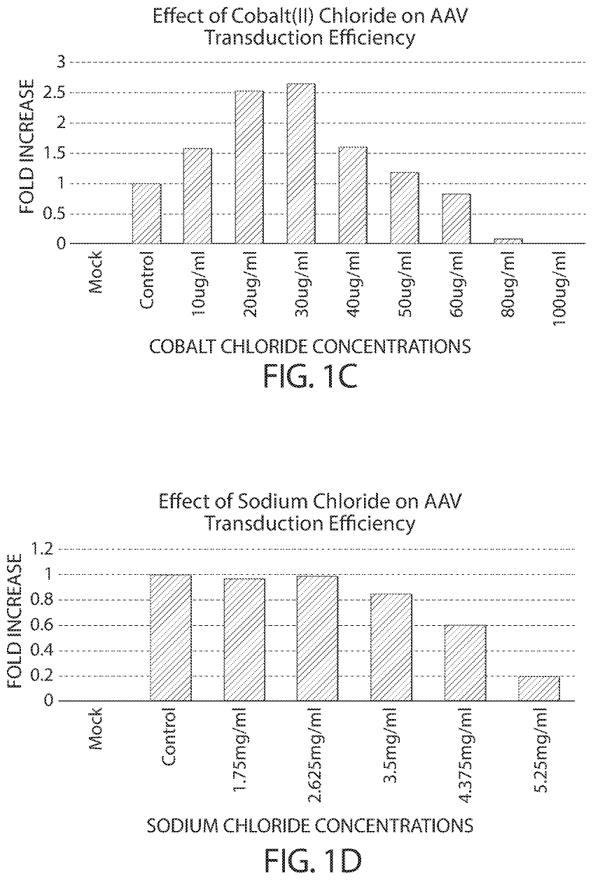

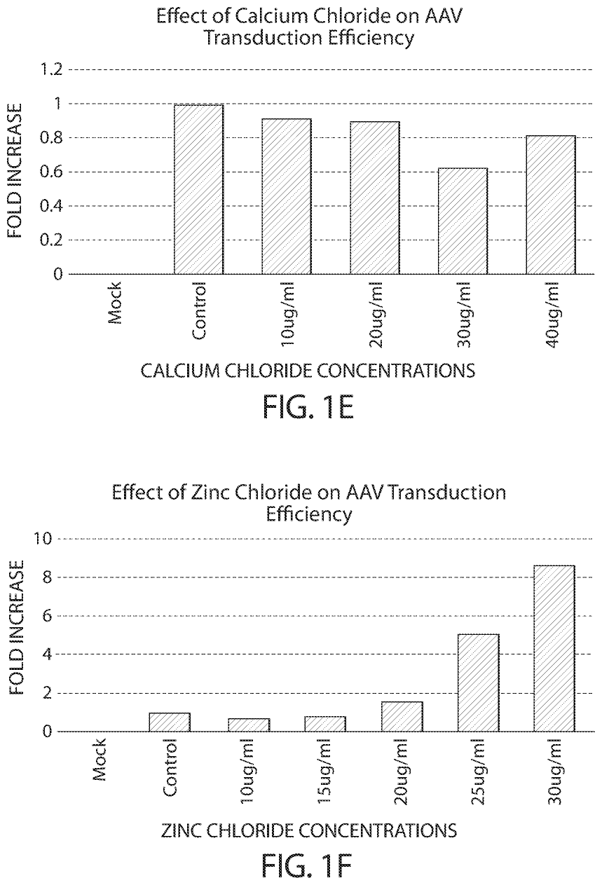

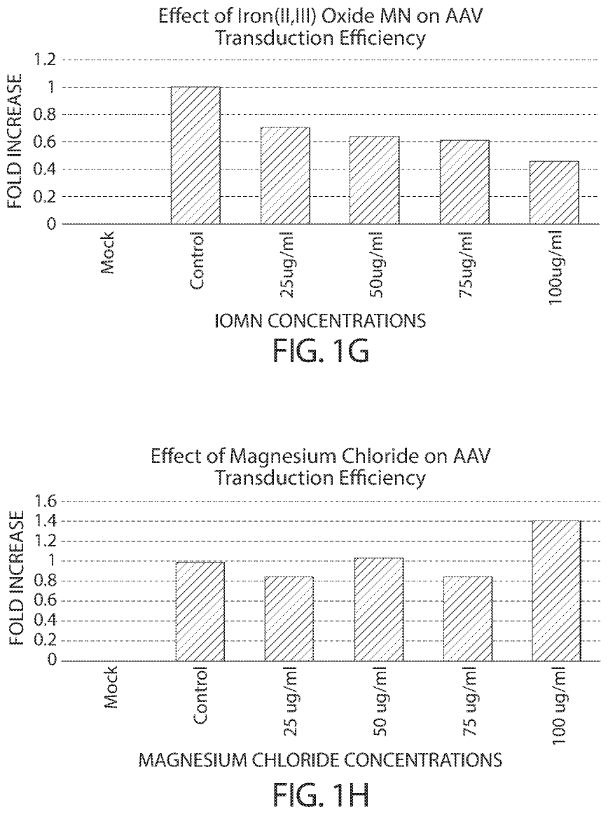

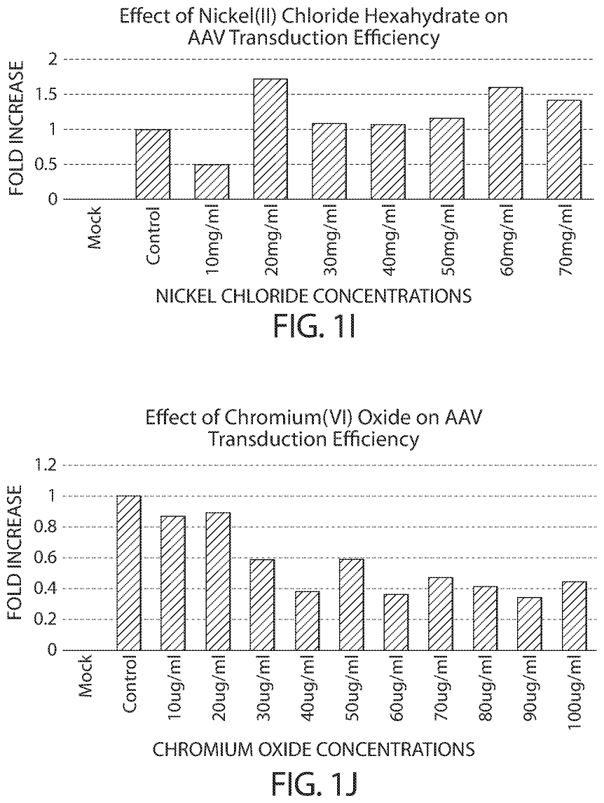

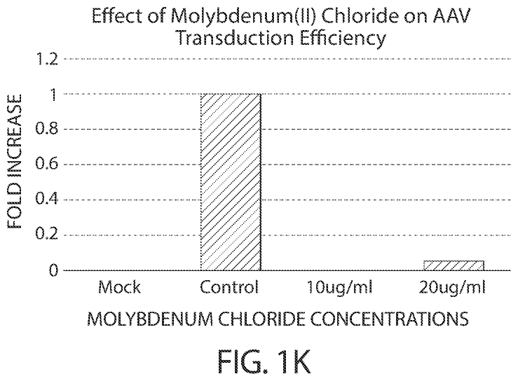

Target blood concentrations can be determined by conducting in vitro experiments in which cells, which are similar or representative of in vivo target tissue, are infected with rAAV and treated with varying concentrations of essential metal elements. The concentration at which the greatest improvement of AAV transduction efficiency is observed (e.g., expression of a transgene comprised in the rAAV) can be treated as the target blood concentration for that essential metal element. For example, based on FIG. 1H, the target blood concentration of magnesium chloride can be determined to be 100 μg/ml; or based on FIG. 1F, the target blood concentration of zinc chloride can be determined to be 30 μg/ml, or based on FIG. 1C the target blood concentration of cobalt chloride can be determined to be 20-30 μg/ml (e.g., 25 μg/ml); or, based on FIG. 1I, the target blood concentration of nickel chloride hexahydrate can be determined to be 200 μg/ml. Concentrations of elemental metal can be calculated based on molecular weights of elements present in the metallic compound. For example, based on a target blood volume of 30 μg/ml of zinc chloride, a target blood concentration of 14 μg/ml of elemental zinc is calculated as the product of the target blood concentration of zinc chloride (30 μg/ml) and the molecular weight of zinc (65 30 g/mole), divided by the molecular weight of zinc chloride (136 g/mole).

Table 1 provides non-limiting examples of target blood concentrations of some essential metal elements in their various forms. In some embodiments, the target blood concentration for zinc is 10-18 μg/ml (e.g., 10-18, 11-17, 12-16, 13-15, 11, 12, 13, 14, 15, 16, or 17 μg/ml). In some embodiments, the target blood concentration for zinc is 14 μg/ml. In some embodiments, the target blood concentration for magnesium is 20-32 μg/ml (e.g., 20-32, 22-30, 24-28, 23-25, 20, 21, 22, 23, 24, 25, 26, 27, 28, 29, 30, 31, or 32 μg/ml). In some embodiments, the target blood concentration for magnesium is 26 μg/ml. In some embodiments, the target blood concentration for cobalt is 8-16 μg/ml (e.g., 8-16, 8-15, 10-14, 11-13, 9, 10, 11, 12, 13, 14, 15, or 16 μg/ml). In some embodiments, the target blood concentration for cobalt is 12 μg/ml. In some embodiments, the target blood concentration for nickel is 5-13 μg/ml (e.g., 5-13, 6-12, 7-11, 8-10, 5, 6, 7, 8, 9, 10, 11, 12, or 13 μg/ml). In some embodiments, the target blood concentration for nickel is 9 μg/ml. It is to be understood that target blood concentrations for essential metal elements will depend on the cell type that is used in vitro to represent the cells or tissue to be targeted in vivo. For example, the target blood concentration of zinc for targeting the brain of a subject may be different from the target blood concentration of zinc for targeting the liver. The target blood concentration of zinc may also be different for targeting neurons of the hippocampus, compared to the target blood concentration of zinc for targeting astrocytes of the subject.

Total blood volume for a subject can be estimated based on the average known total blood volume of a particular age of a particular subject. For example, the total blood volume of an adult mouse can be estimated based on known values to be approximately 2 ml. Similarly, in some embodiments, the total blood volume of an adult human being can be estimated based on known values to be approximately 5 liters. Methods of estimating a subject's total blood volume based on a subject's weight, sex, height, body type or tone, or any combination of these factors are known in the art. For example, the total blood volume (in ml) of an adult male can be calculated using Nadner's equation as ((0.006012×Height³)/(14.6×Weight))+604, wherein the height is entered in inches and the weight is entered in pounds (Nadler S B, Hidalgo J H, Bloch T. Prediction of blood volume in normal human adults. Surgery. 1962; 51:224-232). Further, Gilcher's rule of five can be used to account for different body types assuming that muscular men have 75 ml of blood per kilogram of body weight, normal men have 70 ml of blood per kilogram of body weight, thin men have 65 ml of blood per kilogram of body weight, and obese men have 60 ml of blood per kilogram of body weight. The total blood volume (in ml) of an adult female may be calculated as ((0.005835×Height³)/(15×Weight))+183, wherein the height is entered in inches and the weight is entered in pounds. Gilcher's rule of five may be applied to female subjects as well, wherein muscular women have 70 ml of blood per kilogram of body weight, normal women have 65 ml of blood per kilogram of body weight, thin women have 60 ml of blood per kilogram of body weight, and obese women have 55 ml of blood per kilogram of body weight.

For growing adults (e.g., infants and children) the following estimates may be used to calculate total blood volumes: a newborn that is only 15 to 30 minutes old will have an average blood volume of 76.5 ml of blood per kilogram; a newborn that is 24 hours old has 83.3 ml per kilogram, a three month old has 87 ml per kilogram, a six month old has 86 ml per kilogram, a child one to six years old has 80 ml per kilogram, a ten year old has 75 ml per kilogram, a fifteen year old has 71 ml per kilogram; as teens reach adult sizes and proportions, their blood volume will be the same as that of adults.

In some embodiments, a subject is administered an essential metal element in an amount that is at least sufficient to achieve a target tissue concentration of that element. Doses of essential metal elements for achieving target tissue concentrations can be estimated based on the mass, volume, and/or density of the target tissue, which in turn can be estimated using a number of techniques (e.g., imaging such as magnetic resonance imaging).

In some embodiments, a subject is administered an essential metal element in an amount that is at least sufficient to achieve a target extracellular concentration of that element. Experiments (as described herein) suggests that the extracellular concentrations of metal elements control or effect rAAV transduction. Therefore, in some embodiments, a subject is administered an essential metal ion to reach a target concentration in the extracellular space of a target tissue (e.g., a tumor, or a diseased organ or tissue). Diseases and conditions that involve dysfunctional metal element transport are described above. In addition to tissues involving such diseases, any tissue in which rAAV therapy is targeted can be administered any one or more essential metal elements as disclosed herein. Extracellular target concentrations of metal elements can be achieved by administering a dose of metal element that takes into account the target concentration based on experimental data (e.g., in vitro data showing an optimal concentration for improving rAAV transduction) and the extracellular space volume. Methods of estimating extracellular space volume are known in the art. For example, imaging using fluorescent dye partitioning can be used (see e.g., Zhang and Verkman, Biophys J. 2010 Aug. 9; 99(4): 1284-1291, which is incorporated herein by reference in its entirety). In some embodiments, bioelectrical impedance measurements can be used to measure extracellular space as described by Miholic et al. (Clin Investig (1992) 70: 600. https://doi.org/10.1007/BF00184802). Another method of calculating extracellular space volume is based on the body surface area as described by Filler and Huang (CJASN April 2011, 6 (4) 695-696). Yet another way to measure extracellular space volume is one that is useful to measure any body fluid compartment, which is by measuring the volume of any fluid compartment within the body by injecting or infusing a marker substance that will equilibrate (diffuse freely to a uniform concentration) throughout this compartment. Volume will equal mass divided by concentration (V=M/C), which would equal to (M−MU)/C (where V is the volume of the body fluid compartment, M is the mass of marker injected, MU is the mass of marker lost in the urine during equilibration and C is the measured concentration of the marker. MU is usually calculated from CU, the concentration of marker lost in the urine and VU, the volume of the urine thus: MU=CU·VU).

In some embodiments, a target tissue concentration of any one of the metal elements described herein is 0.1-200 μg/ml (e.g., 0.1-200 μg/ml, 0.1-100 μg/ml, 1-100 μg/ml, 5-90 μg/ml, 10-100 μg/ml, 20-50 μg/ml, 30-50 μg/ml, 40-50 μg/ml, 20-40 μg/ml, 15-30 μg/ml, 0.1-1 μg/ml, 0.1-10 μg/ml, 0.1-20 μg/ml, 10-20 μg/ml, 20-25 μg/ml, 25-30 μg/ml, 35-40 μg/ml, 15-20 μg/ml, or 100-200 μg/ml). In some embodiments, a target tissue concentration of any one of the metal elements described herein is around 0.1 μg/ml, around 1 μg/ml, around 5 μg/ml, around 10 μg/ml, around 15 μg/ml, around 20 μg/ml, around 25 μg/ml, around 30 μg/ml, around 35 μg/ml, around 40 μg/ml, around 50 μg/ml, around 60 μg/ml, around 70 μg/ml, around 80 μg/ml, around 90 μg/ml, or around 100 μg/ml.

In some embodiments, a target extracellular concentration of any one of the metal elements described herein is 0.1-200 μg/ml (e.g., 0.1-200 μg/ml, 0.1-100 μg/ml, 1-100 μg/ml, 5-90 μg/ml, 10-100 μg/ml, 20-50 μg/ml, 30-50 μg/ml, 40-50 μg/ml, 20-40 μg/ml, 15-30 μg/ml, 0.1-1 μg/ml, 0.1-10 μg/ml, 0.1-20 μg/ml, 10-20 μg/ml, 20-25 μg/ml, 25-30 μg/ml, 35-40 μg/ml, 15-20 μg/ml, or 100-200 μg/ml). In some embodiments, a target extracellular concentration of any one of the metal elements described herein is around 0.1 μg/ml, around 1 μg/ml, around 5 μg/ml, around 10 μg/ml, around 15 μg/ml, around 20 μg/ml, around 25 μg/ml, around 30 μg/ml, around 35 μg/ml, around 40 μg/ml, around 50 μg/ml, around 60 μg/ml, around 70 μg/ml, around 80 μg/ml, around 90 μg/ml, or around 100 μg/ml.

In some embodiments, an essential metal element is administered so that it remains below a level that may be toxic to a subject. Toxicity will depend on the particular metal element and on the level of metal element in the blood and/or body, but also on the length in time of exposure to the metal element. In general, the lower the concentration of the metal element, the longer the element can be tolerated without manifesting toxic side effects. Accordingly, in some embodiments, a subject is administered a sub-toxic level of one or more essential metal elements. A sub-toxic level of essential metal element is a level that does not result in any toxic effects in the subject over the period of time that the subject is administered a regimen of essential metal elements. In some embodiments, a sub-toxic level is below the level of metal element that is known to cause toxicity even in the long-term (e.g., longer than the regimen of essential metal elements).

In some embodiments, an essential metal element is administered so that it both improves the transduction efficiency of rAAV particles and also provides direct therapeutic benefit to the target cells or tissue (e.g., cancer cells or tissue), for example by providing toxicity to cancer cells, or rectifying an underlying metal element deficiency that is involved in disease. For example, selective intracellular zinc deficiency has been found to be sufficient to augment expression of pro-angiogenic and pro-metastatic cytokines in established prostate cancer cells (Prostate. 2008 Sep. 15; 68(13): 1443-1449). For example cell-penetrable zinc chelators are used as a therapy for cancers (see e.g., Hashemi et al., Cytotoxic effects of intra and extracellular zinc chelation on human breast cancer cells; Eur J Pharmacol. 2007 Feb. 14; 557(1):9-19. Epub 2006 Nov. 10).

In some embodiments, an essential metal element is administered to a subject enterally (e.g., oral, sublingual, or rectal). In some embodiments, an essential metal element is administered to a subject parenterally (e.g., percutaneously, subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs). In some embodiments, an essential metal element is administered by injection into the hepatic artery or the portal vein.

In some embodiments, a rAAV particle is administered to a subject enterally (e.g., oral, sublingual, or rectal). In some embodiments, a rAAV particle is administered to a subject parenterally (e.g., percutaneously, subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs). In some embodiments, a rAAV particle is administered by injection into the hepatic artery or the portal vein.

In some embodiments, when more than one essential metal elements are administered to a subject, they are administered simultaneously. In some embodiments, multiple essential metal elements are comprised in a single composition that can be administered to a subject. Such a composition may comprise different essential metal elements in different concentrations or proportions relative to each other.

In some embodiments, one or more essential metal elements are administered to a subject prior to administration of rAAV. In some embodiments, a subject receives multiple administrations of one or more essential metal elements prior to receiving rAAV. In some embodiments, a subject receives rAAV and essential metal elements at the same time (within a few minutes, e.g., within 2 minutes, within 5 minutes, within 10 minutes, within 20 minutes, within 30 minutes, or within 60 minutes). In some embodiments, one or more essential metal elements are administered to a subject only after a subject has received rAAV (e.g., more than 1 min after, 2 minutes after, 5 minutes after, 10 minutes after, 1 hour after, 2 hours after, 4 hours after, or 6 hours after receiving rAAV virus). In some embodiments, one or more essential metal elements are administered to a subject a substantial time after receiving rAAV (e.g., after 6 hours, after 8 hours, after 12 hours, after 24 hours, after 2 days, after 3 days, after 4 days, after 5 days, after 6 days, after 1 week, after 2 weeks, after 3 weeks, or after 4 weeks). In some embodiments, a subject is administered one or more essential metal elements both before and after receiving a rAAV. In some embodiments, a subject is administered multiple doses (e.g., 2, 3, 4, 5, 6, 7, 8, 9, 10, or up to 12 doses) of one or more essential metal elements after receiving rAAV. For example, a subject may be administered one or more essential metal elements within 5 minutes of receiving a rAAV, and then again administered one or more essential metal elements 1 week after receiving the rAAV virus. In some embodiments, a subject is administered multiple doses (e.g., 2, 3, 4, 5, 6, 7, 8, 9, 10, 12, 15, 20, 28, 30, 60, 120, or up to 240 doses) of one or more essential metal elements before receiving rAAV. For example, a subject may be administered one or more essential metal elements every day starting 1 week before receiving rAAV. In some embodiments, a subject is administered multiple doses (e.g., 2, 3, 4, 5, 6, 7, 8, 9, 10, or up to 12 doses) of one or more essential metal elements both before and after receiving rAAV. For example, a subject may be administered one or more essential metal elements every day starting 1 week before receiving rAAV, and every day for one to up to four weeks after receiving rAAV.

In some embodiments, one or more essential metal elements and rAAV to be administered to a subject are comprised in the same composition or formulation.

### Administering Essential Metal Elements for Treating Cancer

As shown in the Examples, some cells have a lower tolerance to metal elements, e.g., cells with a reduced concentration of metal element transporters. Such cells are more susceptible to metal element-induced cell death, and undergo cell death at a lower metal element concentration compared to other cells. This phenomenon can be used to induce death in cancerous cells. Accordingly, provided herein is a method comprising administering to a subject having cancer a metal element (e.g., an essential metal element) at a concentration that induced cell death. In some embodiments, a metal element is administered directly to a target tissue (e.g., cancerous tissue or a tumor) to compartmentalize the volume in which metal element concentration is raised.

Recombinant AAV (rAAV) and Methods of Packaging rAAV

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle: Rep78, Rep68, Rep52 and Rep40. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) as disclosed herein may comprise a viral capsid and a nucleic acid vector, which is encapsidated by the viral capsid. In some embodiments, rAAV may be an empty capsid and does not comprise any transgenes. In some embodiments, rAAV may be self-complementary (scAAV). In some embodiments, rAAV may be chimeric (e.g., containing a capsid protein comprising amino acids of different serotypes, or a rep gene comprising base pairs of different serotypes). In some embodiments, a rAAV is pseudotyped (e.g., comprising a capsid protein of one serotype and a rep gene of another serotype).

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

In some embodiments, a gene of interest encodes a detectable molecule. In some embodiments, a detectable molecule is a fluorescent protein, a bioluminescent protein, or a protein that provides color (e.g., β-galactosidase, β-lactamases, β-glucuronidase and spheriodenone). In some embodiments, a detectable molecule is a fluorescent, bioluminescent or enzymatic protein or functional peptide or functional polypeptide thereof. In some embodiments, a gene of interest encodes a therapeutic protein or therapeutic RNA. In some embodiments, a therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, an anticoagulant, a nuclease, guide RNA or other nucleic acid or protein for gene editing.

In some embodiments, the nucleic acid vector comprised in a rAAV is between 4 kb and 5 kb in size (e.g., 4.2 to 4.7 kb in size). Any nucleic acid vector described herein may be encapsidated by a viral capsid, such as an AAV5 or AAV6 capsid or any other serotype, which may comprise a chimeric capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

As mentioned above, in some embodiments, the nucleic acid vector comprises (1) one or more heterologous nucleic acid regions comprising a sequence encoding an RNA, protein or polypeptide of interest, (2) one or more nucleic acid regions comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences. In some embodiments, the nucleic acid vector comprises one or more heterologous nucleic acid regions comprising a sequence encoding an RNA, protein or polypeptide of interest operably linked to a control element (e.g., a promoter), wherein the one or more heterologous nucleic acid regions are flanked on each side with an ITR sequence. Such a nucleic acid vector is herein also referred to as AAV-ITR containing one or more genes of interest. The ITR sequences can be derived from any AAV serotype (e.g., serotype 1, 2, 3, 4, 5, 6, 7, 8, 9, 10, 11, 12, or 13) or can be derived from more than one serotype.

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

Genbank reference numbers for sequences of AAV serotypes 1, 2, 3, 3B, 4, 5, 6, 7, 8, 9, 10, 11, 12, and 13 are listed in patent publication WO2012064960, which is incorporated herein by reference in its entirety for the purpose of incorporating Genbank reference numbers, as well as for any other purpose.

Capsid of a rAAV particle may comprise capsid proteins VP1, VP2, and VP3. In some embodiments, a capsid comprises only two capsid proteins.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter. For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter (e.g. chicken (3-actin promoter) and human elongation factor-1 α (EF-1α) promoter.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. Non-limiting examples of such promoters that may be used include airway epithelial cell-specific promoters.

Synthetic promoters are also contemplated herein. A synthetic promoter may comprise, for example, regions of known promoters, regulatory elements, transcription factor binding sites, enhancer elements, repressor elements, and the like.

Recombinant AAV may be of any AAV serotype, including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). Pseudotyping refers to using the capsid of one serotype and the genome of another serotype, or the mixing of a capsid and genome from different viral serotypes. These serotypes are denoted using a slash, so that AAV2/5 indicates a virus containing the genome of serotype 2 packaged in the capsid from serotype 5.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV can be packaged and subsequently purified.

In some embodiments, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprises rep and/or cap genes that encode the Rep protein and/or Cap proteins for use in a method described herein. In some embodiments, the packaging is performed in vitro.

In some embodiments, a plasmid containing comprising the gene of interest is combined with one or more helper plasmids, e.g., that contain a rep gene of a first serotype and a cap gene of the same serotype or a different serotype, and transfected into helper cells such that the rAAV is packaged.

In some embodiments, the one or more helper plasmids include a first helper plasmid comprising a rep gene and a cap gene, and a second helper plasmid comprising one or more of the following helper genes: Ela gene, E1b gene, E4 gene, E2a gene, and VA gene. For clarity, helper genes are genes that encode helper proteins Ela, E1b, E4, E2a, and VA. In some embodiments, the cap gene is modified such that one or more of the proteins VP1, VP2 and VP3 do not get expressed. In some embodiments, the cap gene is modified such that VP2 does not get expressed. Methods for making such modifications are known in the art (Lux et al. (2005), J Virology, 79: 11776-87)

Helper plasmids, and methods of making such plasmids, are known in the art and commercially available (see, e.g., pDF6, pRep, pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also know and available. For example pSub201 is a plasmid that comprises the coding regions of the wild-type AAV2 genome (Samulski et al. (1987), J Virology, 6:3096-3101).

An exemplary, non-limiting, rAAV production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus and herpesvirus.

Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV production. The rAAV can then be purified using any method known in the art or described herein, e.g., by iodixanol step gradient, CsCl gradient, chromatography, or polyethylene glycol (PEG) precipitation.

Administration and Dosing of rAAV

As disclosed herein, rAAV is administered to a subject as a means to deliver a transgene. In some embodiments, a particular tissue is targeted. In some embodiments, rAAV of a particular serotype is chosen to target a particular tissue based on its tropism. Tissue tropism of different rAAV serotypes are known in the art and can be tested using cells of different tissues.

In some embodiments, “administering” or “administration” means providing a material to a subject in a manner that is pharmacologically useful.

In some embodiments, the number of rAAV particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiments, rAAV particles of higher than 10¹³ particles/ml are be administered. In some embodiments, the number of rAAV particles administered to a subject may be on the order ranging from 10⁶ to 10¹⁴ vector genomes (vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10¹³, or 10¹⁴ vgs/ml. In one embodiment, rAAV particles of higher than 10¹³ vgs/ml are be administered. The rAAV particles can be administered as a single dose, or divided into two or more administrations as may be required to achieve therapy of the particular disease or disorder being treated. In some embodiments, 0.0001 ml to 10 mls are delivered to a subject. In some embodiments, the number of rAAV particles administered to a subject may be on the order ranging from 10⁶-10¹⁴ vg/kg, or any values therebetween, such as for example, about 10⁶, 10⁷, 10⁸, 10⁹, 10¹⁰, 10¹¹, 10¹², 10¹³, or 10¹⁴ vgs/mg. In some embodiments, the number of rAAV particles administered to a subject may be on the order ranging from 10¹²-10¹⁴ vgs/kg.

When administered with essential metal elements that improve AAV transduction efficiency, fewer rAAV particles are needed to be administered. For example, when administered with essential metal elements, 10¹³ vg/kg may be needed to be administered instead of 10¹⁴ vg/kg without essential metal elements.

### Pharmaceutical Formulations

Provided herein are compositions of essential metal elements and rAAV that can be administered to a subject. In some embodiments, compositions of essential metal elements and rAAV are separate. In some embodiments, essential metal elements and rAAV are formulated in a single pharmaceutical composition.

In some embodiments, the composition comprises a pharmaceutically acceptable carrier. The term “carrier” refers to a diluent, adjuvant, excipient, or vehicle with which the rAAV is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

In some embodiments, a composition of essential metal element is not in the form of what would be considered food. For example, shellfish would not be considered a composition of zinc. In some embodiments, a composition of essential metal element is not in a solid dosage form. In some embodiments, a composition of essential metal element is not a dietary supplement. In some embodiments, a composition of essential metal element is a dietary supplement. In some embodiments, the purpose of administering an essential metal element is to increase the effectiveness of an administered rAAV or rAAV that is to be administered to a subject. In some embodiments, the purpose of administering an essential metal element is to hinder the viability of one or more types of unwanted cells (e.g., a cancer cell). In some embodiments, the purpose of administering an essential metal element to a subject is not as a dietary supplement.

In some embodiments, any one of the methods disclosed herein is used to treat a population of subjects (e.g., patients suffering from a type of cancer or any other disease). In some embodiments, a population of subjects is more than 100 subjects (e.g., more than 100, more than 200, more than 300, more than 400, more than 500, more than 1000, more than 1500, more than 2000, more than 3000, more than 4,000, more than 5,000, more than 10,000, more than 15,000, or more than 20,000 subjects).

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, a formulation or composition of a first administration of an essential metal element is the same as that for a subsequent administration of an essential metal element (e.g., the formulations for a first and a second administration of zinc are for subcutaneous injection). In some embodiments, a formulation or composition of a first administration of an essential metal element is the same as that for a subsequent administration of an essential metal element (e.g., the formulation for a first administration of zinc is for a subcutaneous injection, while the formulation of a second administration is for an oral dose of zinc).

Replication and/or production of rAAV can be measured by measuring the quantity of rAAV, assessed either by measuring the quantity of rAAV genes or proteins (e.g., the rep gene, or one or more capsid proteins). Methods of measuring quantity of genes and proteins in cellular or tissue samples are well known in the art, and include quantitative PCR, microscopy, or numerous forms of ELISA. Assays for recombinant AAV production are provided in U.S. Pat. Nos. 6,632,670, and 6,153,436, which are incorporated herein by reference in their entirety.

### Improvement of Transduction Efficiency

In some embodiments, administering one or more essential metal elements to a subject who is to receive or who has received rAAV will result in an improvement in the transduction efficiency of the administered rAAV, relative to if the rAAV were administered without essential metal elements. In some embodiments, any one of the methods disclosed herein involve administering one or more essential metal elements in an amount that results in increased improvement in AAV transduction efficiency in vivo. In some embodiments, any one of the methods disclosed herein involve administering one or more essential metal elements in an amount that results in increased improvement in AAV transduction efficiency in an in vitro context, even if administered in vivo. For example, the increase in transduction efficiency for an in vivo administration of rAAV and essential metal elements can be determined by introducing the combination of rAAV and essential metal elements to cells cultured in vitro, and comparing the transduction efficiency to cells that are introduced only to rAAV without essential metal elements. A cell used for in vitro measurement of AAV transduction efficiency may be cultured in a culture dish, or within an organoid.

Transduction efficiency can be measured by allowing rAAV of a fixed multiplicity of infection (MOI) to infect cells and measuring the amount of expressed RNA or protein from the genetic load that is delivered by the rAAV. For example, a fluorescent (e.g., EGFP) or luciferase gene can be delivered using rAAV and after a certain time (e.g., 24h, or 48h), and either fluorescent or luciferase RNA expression or luciferase protein can be measured using one of numerous techniques known in the art (e.g., cell fractionation, polymerase chain reaction, microscopy, and/or luciferase enzyme assays).

In some embodiments of any one of the methods provided herein, AAV transduction efficiency is improved by administration of essential metal elements by a factor of 1.2-100 times (e.g., 1.2-100, 1.3-5, 1.4-5, 1.5-2, 1.2-10, 10-20, 10-15, 12-18, 15-20, more than 1.5, more than 2, more than 3, more than 4, more than 5, more than 7, more than 8, more than 10, more than 12, more than 14, more than 16, or more than 20 fold), compared to when no essential metal elements are administered. In some embodiments, the difference between the conditions of when rAAV is administered or tested without essential metal elements and with essential metal elements is that the condition without essential metal elements consists of only basal levels of essential metal elements (e.g., that which might be contained in cell culture media).

### Methods of Treatment and Methods of Administering a Gene Therapy

Any of the methods provided herein may be used in gene therapy to treat a condition or disease which may be treated by delivering a therapeutic gene to target cells, or tissue. In some embodiments, a therapeutic gene encodes a therapeutic protein or therapeutic RNA. In some embodiments, a therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, an anticoagulant, a nuclease, guide RNA or other nucleic acid or protein for gene editing.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV may be an amount of the rAAV that is capable of transferring an expression construct to a host organ, tissue, or cell. A therapeutically acceptable amount may be an amount that is capable of treating a disease. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

### Ex Vivo Applications

In some embodiments, any one of the methods disclosed herein can be used to deliver one or more genes to cells and then delivering those cells as a therapy to a subject. In some embodiments, cells that are infected with rAAV and treated with essential metal elements as disclosed herein are autologous. In some embodiments, cells that are infected with rAAV and treated with essential metal elements as disclosed herein are allogenic. For example, any one of the methods disclosed herein can be used to deliver a transgene to a subject's own cells (e.g., to reprogram or differentiate a cell) and administer infected cells back to the subject. In some embodiments, any one of the methods disclosed herein can be used to reprogram a subject's own cells in connection with chimeric antigen receptor T cell (CAR-T) therapy (see e.g., US20140271635A1, WO2016028896A1, and US20160340406A1, each of which is incorporated by reference in its entirety).

### In Vitro Applications

Methods disclosed herein can be used for in vitro applications as well, e.g., in the lab to study rAAV effects or to develop rAAV.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Essential Metal Elements Increase the AAV Transduction Efficiency in Cells Infected with rAAV

The following study was carried out to evaluate the role of essential metal elements in AAV infection of cells and transgene expression.

In an initial set of experiments, HeLa cells were infected with self-complementary AAV2 (scAAV2) comprising nucleic acid encoding EGFP (scAAV2-EGFP) at a multiplicity of infection of 200 viral genomes (vgs) per cell. Thereafter, various essential metal elements were added to the cell cultures at different concentrations. Transduction efficiency was measured 48 hours after infection, by evaluating the expression of EGFP using fluorescent microscopy. Images were analyzed using ImageJ software.

The results showed a dose-dependent increase in transgene expression (FIGS. 1A-1K), with a maximal increase with approximately 100 μg/ml of magnesium chloride (˜2 fold compared to when no essential metal element was added), 30 μg/ml of zinc chloride (˜8 fold compared to when no essential metal element was added), 25 μg/ml of cobalt chloride (˜2 fold compared to when no essential metal element was added), and 20 μg/ml of nickel chloride (˜3 fold compared to when no essential metal element was added).

Surprisingly, when cells were co-treated with multiple essential metal elements, the transduction efficiency was improved in a way that was more than additive of the effects of treating the cells with only each of the essential metal elements. For example, treating the cells with zinc chloride and cobalt chloride, zinc chloride and nickel chloride, or zinc chloride and magnesium chloride, led to ˜15 fold increase in transduction efficiency compared to when no essential metal element was added (FIGS. 2A-2B). Similar levels of augmentation in transduction efficiency was observed using other AAV serotypes, e.g., AAV1, AAV3, AAV4, AAV5, and AAV6.

### Example 2: Effect of Essential Metal Elements in Mice Administered with rAAV

To evaluate the effect on AAV transduction efficiency in vivo, mice are administered rAAV comprising a EGFP transgene, and then a composition of one or more metal elements either through the portal vein or hepatic artery. Control mice are either administered rAAV alone, or a control composition comprising all other components of the composition containing the rAAV, with the exception of the rAAV.

Essential metal elements are administered once immediately after administration of the rAAV in test mice, and a second time a week after they are administered the rAAV. In order to maintain the highest essential metal element concentration in the blood and target tissue, the essential metal elements are administered by injection into the hepatic artery or portal vein. The quantities of each essential metal element to be administered to a mouse is determined by multiplying the concentration of that essential metal element that led to the highest transduction efficiency in in vitro experiments, and the total volume of blood in a mouse, assumed to be approximately 2 ml. For example, to achieve a concentration of 30 μg/ml of zinc chloride in the blood of a mouse, 60 μg of zinc chloride is administered to the mouse. This 60 μg of zinc chloride can be provided in a volume of zinc chloride composition that will allow proper solubility of zinc chloride in buffer.

Two weeks after administration of the rAAV, the mice are euthanized and the target tissues harvested to evaluate transgene expression. EGFP is used as the transgene.

### Example 3. Mechanism of Action of Metal Ions on Transduction of rAAV Particles

As described above, at a final concentration of 30 μg/ml, an ˜8-fold increase was observed in HeLa cells. However, at a final concentration exceeding 30 μg/ml, Zn²⁺ also induced significant cytotoxicity, an observation consistent with a previously published report (Exp. Biol. Med., 235: 741-750, 2010). In subsequent studies, similar results were obtained with HEK293 cells. However, when these studies were extended to include a human hepatocellular carcinoma (HCC) cell line, Huh7, ˜7-fold increase in the transgene expression was observed, but in the presence of only 15 μg/ml of Zn²⁺, and concentrations exceeding 20 μg/ml of Zn²⁺ were found to be cytotoxic to Huh7 cells (FIG. 3).

Further mechanistic studies to gain a better understating of these phenomena revealed that the increase in transduction occurred due to higher levels of extracellular levels of Zn2⁺. The effect of metal ion (e.g., zinc) on the transduction efficiency of AAV vectors may be due to free floating zinc, because the increase in transduction occurs when the cell has taken up zinc to the point where extracellular accumulation occurs. In Huh7 cells transfected with a ZIP14 gene, there is a reduction in the zinc-induced improvement of rAAV transduction efficiency. ZIP14 is a metal ion transporter that primarily transports zinc into cells.

These findings support use of metal ions in certain diseased tissue. For example, hallmark decreases in intracellular Zn2+ levels have been reported in HCC patients dating back to the 1970s. Numerous population studies have reported a significant decrease (55-75%) in Zn2+ levels in HCC tissues, compared with healthy liver tissues (Cancer Bio. & Ther., 15: 353-360, 2014). It has also been reported that the decrease in Zn2+ levels in HCC tissues is due to under-expression of an essential metal ion transporter, SLC39A14 (ZIP14), and that ZIP14 plays a major role in the transport of Zn2+ in liver cells (J. Gastrointest. Cancer, 43: 249-257, 2012).

Therefore, targeting ZIP14-deficient HCC tumors would be ideal for Zn2+ infusions due to their tendency to prevent zinc uptake, and therefore, increase the level of environmental Zn2+. To test this hypothesis, Huh7 cells were either mock-transfected, or transfected with a recombinant plasmid containing a CMV promoter-driven ZIP14-EGFP fusion protein, and were transduced with scAAV2-mCherry vectors under identical conditions. The results showed that ZIP14 over-expressing, but not mock-treated Huh7 cells, evaded Zn2+-induced cytotoxicity at a final concentration of 20 μg/ml.

Recombinant AAV vectors containing the ZIP14 gene are produced that can be used for co-administration strategies with AAV vectors carrying a therapeutic gene such that subsequent Zn2+ infusions directly into ZIP14-deficient HCC tumors. Such strategies are useful especially in view of a negative correlation between ZIP14 expression and patient survival times (J. Trace Elem. Med. Biol., 49: 35-42, 2018) indicating intracellular zinc imbalances may play a role in cancer survival as well. These concerns notwithstanding, AAV vector-mediated delivery and expression of ZIP14 gene would be expected to augment not only Zn2+ uptake, but also Zn2+-mediated increased expression of a therapeutic gene in human liver cancer tissues.

### Example 4. Verification of Improvement of rAAV Transduction Efficiency after Treatment with Zinc Chloride

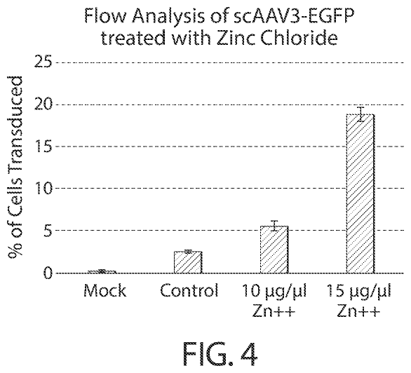

Huh7 cells were infected with self-complementary AAV3 (scAAV3) comprising nucleic acid encoding EGFP (scAAV3-EGFP). Thereafter, zinc chloride was added to the cell cultures at different concentrations. Transduction efficiency was measured by evaluating the expression of EGFP using flow cytometry. FIG. 4 shows that the percentage of cells transduced by the scAAV3-EGFP vector increases with increasing concentration of zinc chloride.

### Example 5. Comparison of Transduction Efficiency Trends and Cell Viability Trends as a Function of Metal Ion Concentrations

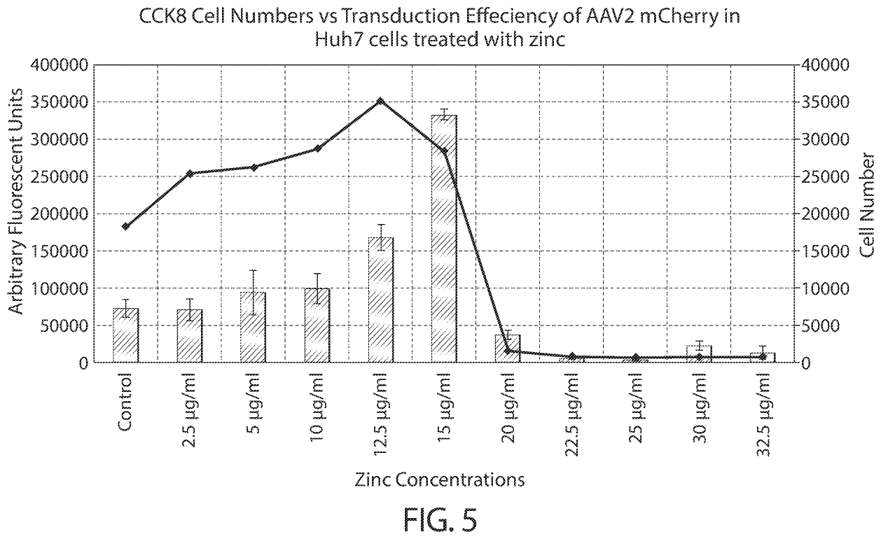

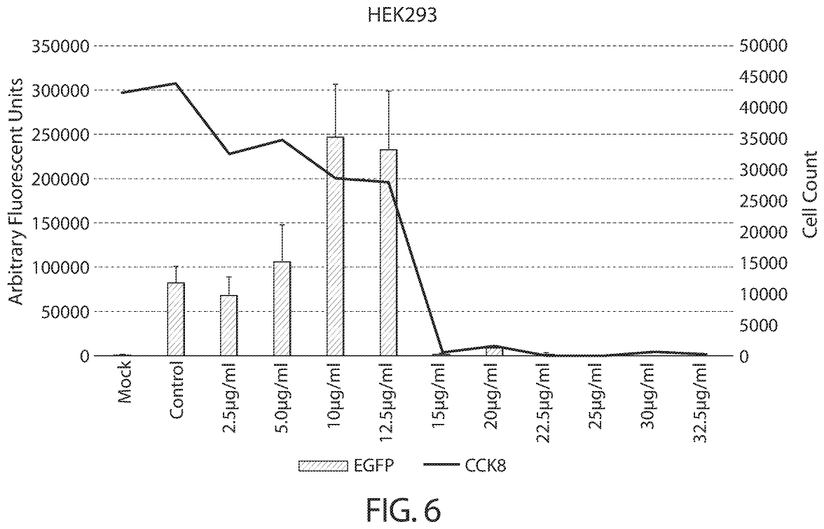

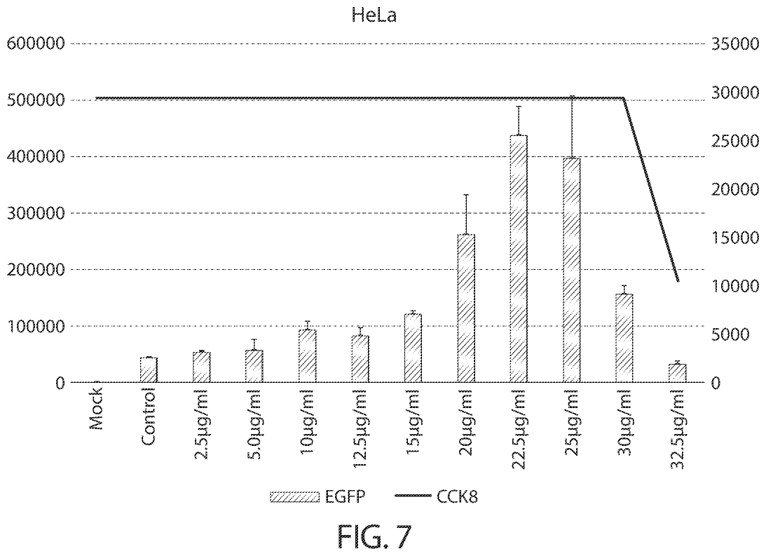

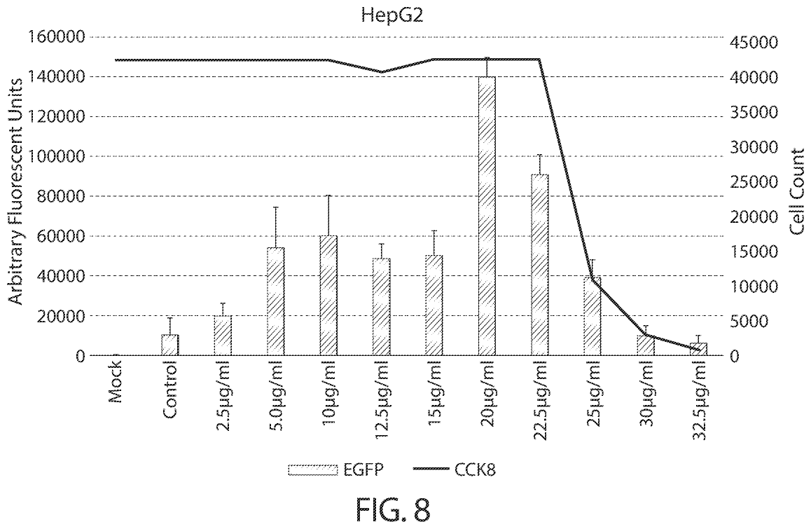

FIG. 5 provides an overlap of AAV transduction efficiency data and cell viability data from experiments in which CCK8 cells were transduced with AAV2 comprising mCherry encoding nucleic acid and treated with varying concentrations of zinc. FIG. 5 shows that the drop in rAAV transduction efficiency correlates with cell viability. FIGS. 6, 7, and 8 show similar data for HEK293 cells, HeLa cells, and HepG2 cells.

### Example 6. Verification of Improvement of rAAV Transduction Efficiency after Treatment with Essential Metal Elements

HeLa cells are infected with self-complementary AAV2 (scAAV2) comprising nucleic acid encoding EGFP (scAAV2-EGFP) at a multiplicity of infection of 200 viral genomes (vgs) per cell as described above. Thereafter, various essential metal elements are added to the cell cultures at different concentrations, and either within the same concentration ranges as described in FIGS. 1A-1K or broader. Transduction efficiency is measured 48 hours after infection, by evaluating the expression of EGFP using fluorescent microscopy. Images are analyzed using ImageJ software. Sodium, potassium, and calcium are tested up to 5.25 μg/ml, and magnesium is tested up to 2.8 μg/ml. The following metal ions elements are tested up to approximately 33 μg/ml: chlorine, manganese, iron, cobalt, copper, zinc, and molybdenum.

### Example 7. Improving Delivery of Factor IX to HepG2 Cells

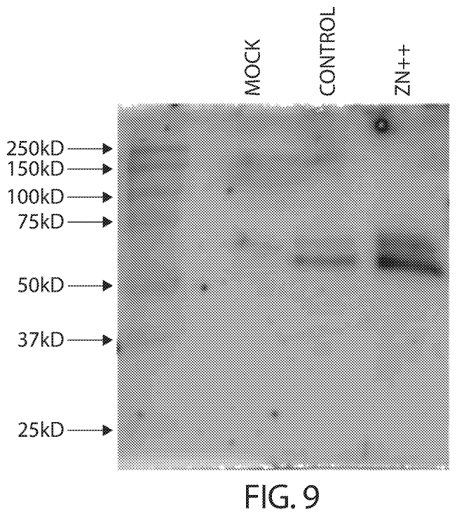

FIG. 9 shows Western blot analysis of Factor IX expression in HepG2 cells infected with AAV3 particles comprising nucleic acid encoding Factor IX gene, either with or without treatment with 20 μg/ml ZnCl₂. HepG2 cells, which express factor IX to a lower degree compared to other cell types, were cultured. They were then either infected with rAAV3 particles carrying nucleic acid encoding Factor IX, or infected with rAAV3 particles carrying nucleic acid encoding Factor IX and treated with 20 μg/ml of ZnCl₂. When level of Factor IX expressed in the cells was measured using Western blot analysis after the cells there harvest 3h post infection. Compared to cells that were not treated with metal elements, those treated with 20 μg/ml of ZnCl₂ shows a greater level of Factor IX expression.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method comprising:
administering to a subject a recombinant adeno-associated virus (rAAV), and
administering to the subject an essential metal element in an amount that is at least equal to a target blood concentration multiplied by the total volume of the subject's blood.

2. A method comprising:
administering to a subject a recombinant adeno-associated virus (rAAV), and
administering to the subject an essential metal element in an amount that results in increased AAV transduction efficiency.

3. The method of claim 1 or claim 2, wherein the subject is administered 2-5 essential metal elements.

4. The method of the preceding claims, wherein the essential metal element is selected from the group consisting of: calcium, magnesium, sodium, chromium, copper, cobalt, iron, manganese, molybdenum, zinc, and nickel.

5. The method of claim 4, wherein the essential metal element is selected from the group consisting of: magnesium, zinc, cobalt, and nickel.

6. The method of any one of claims 4-5, wherein the essential metal element administered to the subject is zinc.

7. The method of any one of claims 4-6, wherein the essential metal element/s administered to the subject comprises zinc.

8. The method of any one of claim 7, wherein the essential metal element/s administered to the subject comprises zinc and cobalt.

9. The method of any one of claim 7, wherein the essential metal element/s administered to the subject comprises zinc and nickel.

10. The method of any one of claim 7, wherein the essential metal element/s administered to the subject comprises zinc and magnesium.

11. The method of any one of the preceding claims, wherein the essential metal element is provided in the form of a chloride salt.

12. The method of any one of claims 1 and 3-11, wherein the subject is administered magnesium and the target blood concentration is approximately 26 μg/ml, wherein the subject is administered cobalt and the target blood concentration is approximately 12 μg/ml, wherein the subject is administered nickel and the target blood concentration is approximately 9 μg/ml, and/or wherein the subject is administered zinc and the target blood concentration is approximately 14 μg/ml.

13. The method of any one of claims 3-12, wherein the essential metal elements are administered simultaneously.

14. The method of any one of claims 3-13, wherein the essential metal elements are comprised in a single composition.

15. The method of any one of the preceding claims, wherein the rAAV is administered simultaneously with the essential metal element/s.

16. The method of claim 15, wherein the rAAV and essential metal element/s are comprised in a single formulation.

17. The method of any one of the preceding claims, wherein the essential metal element/s is administered to the subject after administering the rAAV.

18. The method of claim 17, wherein the essential metal element/s is administered to the subject at least a second time after administering the rAAV.

19. The method of claim 18, wherein the essential metal element/s is administered to the subject 2-5 times after administering the rAAV.

20. The method of claims 17-19, wherein the essential metal element/s is administered to the subject at least one minute after administering the rAAV.

21. The method of claims 17-20, wherein the essential metal element/s is administered to the subject at least one hour after administering the rAAV.

22. The method of claims 17-21, wherein the essential metal element/s is administered to the subject at least one day after administering the rAAV.

23. The method of claims 17-22, wherein the essential metal element/s is administered to the subject at least one week after administering the rAAV.

24. The method of any one of the claims 18-23, wherein the subject is administered an essential metal element/s the second time at least a week after administering an essential metal element/s to the subject the first time.

25. The method of any one of the preceding claims, wherein the essential metal element/s is administered to the subject enterally.

26. The method of claim 25, wherein the enteral administration of the essential metal element/s is oral.

27. The method of any one of claims 1-24, wherein the essential metal element/s is administered to the subject parenterally.

28. The method of claim 27, wherein the essential metal element is administered to the subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

29. The method of anyone of the preceding claims, wherein the essential metal element/s is administered to the subject by injection into the hepatic artery or portal vein.

30. The method of any one of the preceding claims, wherein the rAAV is single stranded or self-complementary.

31. The method of any one the preceding claims, wherein the serotype of the rAAV is 1, 2, 3, 4, 5, 6, 7, 8, 9, 10, 11, 12, or 13.

32. The method of any one of claims 1-33, wherein rAAV is a pseudotype.

33. The method of any one of the preceding claims, wherein the rAAV comprises a chimeric rep gene, or chimeric capsid protein.

34. The method of any one of the preceding claims, wherein the rAAV comprises a therapeutic gene.

35. The method of claim 37, wherein the therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, a Fc-fusion protein, an anticoagulant, a nuclease, guide RNA or other nucleic acid or protein for gene editing.

36. The method of any one of the preceding claims, wherein the subject is a mammal.

37. The method of claim 36, wherein the mammal is a human.

38. The method of claim 36, wherein the subject is an adult human, and the total blood volume is approximately 5 liters.

39. The method of claim 36, wherein the subject is an adult human, and the total blood volume is determined using Nadner's equation as ((0.006012×Height³)/(14.6×Weight))+604, wherein the height is entered in inches and the weight is entered in pounds.

40. The method of claim 37, wherein the mammal is a mouse, and the total blood volume is approximately 2 ml.

41. The method of claim 36, wherein the mammal has a cancer that is characterized by a reduced intracellular level of an essential metal element.

43. The method of claim 41, wherein the cancer is hepatocellular carcinoma that is characterized by hepatic cells with a reduced intracellular zinc level.

43. The method of any one of claims 2-12 and 15-37, wherein administering an essential metal element/s results in a 1.5-20-fold increase in AAV transduction efficiency when compared to when AAV is administered alone.

44. The method of claim 43, wherein the fold increase in AAV transduction efficiency is measured by infecting test cells in an in vitro culture with AAV with and without essential metal elements.

45. A method comprising administering to a subject that has received a rAAV an essential metal element in an amount that is at least equal to a target blood concentration multiplied by the total volume of the subject's blood.

46. A method comprising administering to a subject that has received a rAAV an essential metal element in an amount that results in increased AAV transduction efficiency.

47. A method comprising administering to a subject who has received a rAAV and an essential metal element the essential metal element a second time.

48. A pharmaceutical composition comprising:
a rAAV, and
one or more essential metal elements,
wherein the ratio of rAAV and one or more essential metal elements results in increased AAV transduction efficiency when administered to a subject, or in a target blood concentration/s of the one or more essential metal elements.

49. The pharmaceutical composition of claim 46, further comprising a pharmaceutically acceptable carrier.

50. A method comprising:
administering to a subject a recombinant adeno-associated virus (rAAV) particle, and
administering to the target tissue or organ an essential metal element in an amount that is sufficient to result in a target extracellular concentration of 1-50 μg/ml of the essential metal element in the target tissue or organ.

51. The method of claim 30, wherein the rAAV particles are administered to the target tissue or organ.

52. The method of claim 50, wherein the target extracellular concentration is around 10 μg/ml, 20 μg/ml, 30 μg/ml, or 40 μg/ml.

53. A method comprising:
administering to a subject an essential metal element, wherein the essential metal element is administered in an amount that is sufficient to result in a target extracellular concentration of 1-50 μg/ml of the essential metal element in a target tissue or organ, or in an amount that is at least equal to a target blood concentration multiplied by the total volume of the subject's blood.

54. The method of claim 53, wherein the subject has cancer.

55. The method of claim 53 or 54, wherein the metal element is administered directly to cancer tissue in the subject.

56. The method of any one of claims 53-55, further comprising administering to a subject a recombinant adeno-associated virus (rAAV) particle.

57. The method of any 56, wherein the rAAV particle comprises a nucleic acid encoding a therapeutic gene.

58. The method of claim 56, wherein the therapeutic gene targets a cancer.

59. The method of any one of claims 53-58, wherein the subject is administered 2-5 essential metal elements.

60. The method of any one of claims 53-59, wherein the essential metal element is selected from the group consisting of: calcium, magnesium, sodium, chromium, copper, cobalt, iron, manganese, molybdenum, zinc, and nickel.

61. The method of claim 61, wherein the essential metal element is selected from the group consisting of: magnesium, zinc, cobalt, and nickel.

62. The method of claim 60 or 61, wherein the essential metal element administered to the subject is zinc.

63. The method of any one of claims 60-62, wherein the essential metal element/s administered to the subject comprises zinc.
